(19) United States
(12) Patent Application Publication
(10) Pub. No.: US 2026/0263825 A1
(43) Pub. Date: Sep. 10, 2026

(54) NON-CONTACT PELVIC FLOOR ASSESSMENT AND TRAINING METHOD

(71) Applicant: MEDLANDER MEDICAL TECHNOLOGY INC., Nanjing, Jiangsu (CN)

(72) Inventors: ZHANGYIN MA, Nanjing, Jiangsu (CN); Zhiwei LV, Nanjing, Jiangsu (CN); Zhengyang BI, Nanjing, Jiangsu (CN)

(21) Appl. No.: 19/482,628

(22) PCT Filed: Jun. 7, 2023
(86) PCT No.: PCT/CN2023/098835

§ 371 Date: Nov. 7, 2025

(30) Foreign Application Priority Data
Mar. 10, 2023 (CN) 202310229211.1

(51) Int. Cl. A61N 2/00; A61N 2/02

(52) U.S. Cl. CPC A61N 2/004; A61N 2/02

(57) ABSTRACT

A non-contact pelvic floor muscle real-time detection and feedback system, method, and apparatus, which relates to the technical field of medical instruments is provided, including a processor module, a magnetic field generation module, a magnetic stimulation module, a non-contact pelvic floor detection module, and a bearing module; The present disclosure adopts an electromagnetic wave detection method to detect the pelvic floor muscle state in real-time in a non-contact manner, effectively protecting user privacy, reducing usage costs, and eliminating the risk of cross infection, allowing a large-scale screening. The non-contact electromagnetic wave sensor used can be far away from the human body and monitor the pelvic floor muscle state outside of high-energy pulse magnetic fields, avoiding the risk of damaging electronic detection equipment due to high-energy pulse magnetic fields.

### CROSS REFERENCE TO RELATED APPLICATIONS

This application claims priority to PCT Application No PCT/CN2023/098835, having a filing date of Jun. 7, 2023, which claims priority to CN application No. 202310229211.1, having a filing date of Mar. 10, 2023, the entire contents of both of which are hereby incorporated by reference.

### FIELD OF TECHNOLOGY

The following relates to the technical field of medical instruments, specifically to a non-contact pelvic floor muscle real-time detection and feedback system, method and apparatus.

### BACKGROUND

At present, the consultation rate for pelvic floor problems among women in China is less than one-third. One reason is that screening methods expose women's privacy. The commonly used method for detecting pelvic floor muscle state is contact-based, which requires sensors to be placed in the vagina for detection, such as vaginal electrodes and airbags. This method cannot protect patients' privacy, has high consumables costs, and poses a risk of cross infection. Due to psychological factors, women are unwilling to seek medical consultations in a timely manner, which reduces their willingness to seek medical consultations. Therefore, there is a lack of a non-contact detection and evaluation method that protects women's privacy currently.

In terms of instruments used for pelvic floor examination, magnetic stimulation can penetrate clothing and stimulate women's pelvic floor muscles through high-energy pulse magnetic fields generated by coils, this method can effectively protect women's privacy. However, the current magnetic stimulation system is still in the open-loop stage, that is, there are no sensors compatible with magnetic stimulation that can detect the pelvic floor muscle state during magnetic stimulation, and cannot monitor the pelvic floor muscle state in real-time, which can then be fed back to the input to adjust the magnetic stimulation parameters. This is mainly because current sensors must be tightly attached to the skin outside the pelvic floor muscle or be deeply inserted into the vagina, which exposes the electronic components of the sensors to high-energy magnetic fields and makes them more susceptible to damage. Therefore, there is a lack of a non-contact detection sensor compatible with magnetic stimulation currently.

In addition, it has been proven in practical use that triggering magnetic stimulation by real-time detection of pelvic floor muscle state to mobilize patients' autonomous willingness is significantly more effective than traditional magnetic stimulation. However, in the current process of magnetic stimulation in the industry, high-intensity pulse magnetic fields are applied to stimulate patients' pelvic floor muscles to produce passive contractions, without the need for patients to actively contract. This does not mobilize patients' voluntary willingness and cannot detect their active contractions in real-time, resulting in low patient participation. Therefore, there is a lack of an implementation plan for triggering magnetic stimulation currently.

Therefore, people need a non-contact real-time detection, training, and feedback system for the pelvic floor to solve the above problems.

### SUMMARY

An aspect relates to a non-contact pelvic floor muscle real-time detection and feedback system, method and apparatus to solve the problems proposed in the conventional art.

To achieve the above purpose, the present disclosure provides the following technical solution: a non-contact pelvic floor muscle real-time detection and feedback system, comprising:
a processor module for controlling a magnetic stimulation module and a magnetic field generation module to generate pulse magnetic fields with different parameters;
a magnetic field generation module for generating pulse magnetic fields;
the magnetic field generation module has a void in the center for allowing the electromagnetic waves transmitted by a non-contact pelvic floor detection module to pass through;
a non-contact pelvic floor detection module for detecting pelvic floor muscle movements by non-contact detection methods;
the non-contact pelvic floor detection module comprises an antenna module for transmitting and receiving the electromagnetic waves; the non-contact pelvic floor detection module is placed below the magnetic field generation module, and the antenna module that transmits and receives the electromagnetic waves is aligned with the center of the void of the magnetic field generation module;
a bearing module for carrying patients;
the bearing module has a raised area for positioning; the raised area is an area where magnetic stimulation is concentrated and is also a detection area of the pelvic floor detection module; the raised area can indicate to the patients and enable them to autonomously adjust and align with the corresponding area of the pelvic floor;
a shielding module for shielding the electromagnetic waves; the shielding module is placed between the non-contact pelvic floor detection module and the bearing module, and is used to shield the electromagnetic waves transmitted by the non-contact pelvic floor detection module; the shielding module has a void in the center that allows some electromagnetic waves to pass through;
the antenna module of the non-contact pelvic floor detection module, the void of the shielding module, the void of the magnetic field generation module, and the raised area of the bearing module are located on a straight line. The electromagnetic waves transmitted and received by the antenna module propagate in the space set by this straight line, and the electromagnetic waves propagated to other areas are shielded by the shielding module. Realizing the detection of specific areas of the human pelvic floor, reducing the interference from non-pelvic floor movements and the interference from complex electromagnetic environments.

According to the above technical solution, the non-contact pelvic floor detection module comprises a signal transceiver module, a signal transmission module, and a signal processing module;
the signal transceiver module is used to detect the pelvic floor muscle state by transmitting and receiving signals; the signal transceiver module can use a radar electromagnetic wave transceiver module, which includes a radar electromagnetic wave transmitting antenna and a receiving antenna;
the signal transmission module is used to transmit the received signal; including signal modulation, ADC, and I/Q transmission;
the signal processing module is used to process the signal data transmitted by the signal transmission module;
the non-contact pelvic floor detection module further comprises a real-time display module for displaying the processed signal data in real-time.

According to the above technical solution, the magnetic field generation module comprises a metal coil inside, the instantaneous current generated by the magnetic stimulation module generates a strong pulse magnetic field through the magnetic field generation module, the magnetic field generation module is placed below the bearing module, the magnetic field generation module has a void in the center that facilitates the electromagnetic waves transmitted by the non-contact pelvic floor detection module to pass through.

According to the above technical solution, the processor module controls the operation of the non-contact pelvic floor detection module, receives data from the non-contact pelvic floor detection module, processes the data, and acquires the pelvic floor muscle real-time state; the processor module detects the pelvic floor state in real-time when the pulse magnetic field stimulates the pelvic floor, analyzes the detected data, and adjusts the magnetic stimulation parameters based on the feedback.

According to the above technical solution, the raised area of the bearing module can play the role of indicating positioning and adjust the posture after the patient sits up, such that making the perineum feel the raised area, realizing the function of magnetic stimulation to accurately stimulate the pelvic floor area and the function of the detection module to accurately detect the pelvic floor area.

A non-contact pelvic floor muscle real-time detection and feedback method, characterized in that, comprising following steps:
S1: generating instantaneous current by a magnetic stimulation module and generating a pulse magnetic field by a magnetic field generation module;
S2: performing a non-contact detection of the pelvic floor by transmitting and receiving electromagnetic waves through a non-contact pelvic floor detection module;
S3: acquiring the detected pelvic floor state data by a processor module, and performing analysis and detection on it;
S4: adjusting magnetic stimulation parameters based on the analysis and detection results from S3.

According to the above technical solution, in steps S2 and S3, performing the non-contact pelvic floor detection and analyzing the pelvic floor state comprising the following steps:
step one:
the signal transceiver module transmits a signal, which touches the pelvic floor area, and generates an echo reflection that is received by the signal transceiver module and transmitted by the transmission module; one way is to transmit the electromagnetic waves by the radar electromagnetic wave transmission module, when the incident electromagnetic waves touch the skin tissue outside the pelvic floor muscle, they generate echoes and reflect back, which are received by the radar receiving antenna, after signal modulation, they are converted into digital signals raw data by the radar's ADC module and transmitted in the form of I/Q signals, wherein, the data format of I/Q signals is I+iQ, I is the real part and Q is the imaginary part; step two:
the signal processing module preprocesses the signals transmitted by the signal transmission module; specifically, one method is to preprocess the obtained raw signal raw data, and after performing a Range Fourier Transform (FFT) on it, the resulting data is signal X={X₁, X₂, . . . , X_{N}}, wherein, X₁, X₂, . . . , X_{N} represent the data from 1 to N distance gates obtained by performing the Range Fourier Transform on the received signal X, representing the radar echo signal values detected from different distances, the size of N values is positively correlated with the number of distance gates;
step three:
the signal processing module processes the preprocessed data and restores the true signal of pelvic floor muscle movement in the preprocessed signal; specifically, one method is sliding window filtering processing, due to the fact that the object touched by electromagnetic waves after transmission includes not only the target pelvic floor area, but also other static components such as walls and seats, in order to ensure that the radar phase changeΔ φ is positively correlated with the pelvic floor muscle movement change Δ d, it is necessary to remove the static component in signal X, namely: X=Aₘe^{jWm}+Aₛe^{jWs}.
wherein, Aₘ represents the amplitude of the dynamic signal component, Aₛ is the amplitude of the static signal component, Wₘ is the phase of the dynamic signal component, Wₛ is the phase of the static signal component;
selecting a sliding window for real-time signal processing, the window length WinLen needs to be set empirically based on the radar sampling rate;
performing mean removal on the signal values within each window, i.e. X′ᵢ=Xᵢ−mean (X_{i-WinLen+1}:Xᵢ);
wherein, mean (X_{A}:X_{B}) represents the mean of the A-th to B-th data in array X;
step four:
using feature modeling and classification methods for feature extraction to determine the state of pelvic floor muscle; after the mean filtering, the low-frequency components, i.e. the static component Aₛe^{jWs}, in the signal X=Aₘe^{jWm}+Aₛe^{jWs} are basically filtered out, after passing through the filter, Aₘ in the dynamic component Aₘe^{jWm} remains basically unchanged, but its phase Wₘ undergoes sudden changes, resulting in irregular changes in the waveform, therefore, the calculated radar phase changeΔ φ is not correlated with the pelvic floor muscle movement change Δ d.

In conventional processing methods, it is not possible to recover the signal waveform of the true movement of the pelvic floor muscle, and the waveform cannot correspond to the pelvic floor muscle movement, and when the pelvic floor muscle remain stationary and contracted for a long time, the filtered waveform is prone to jumping, which affects the test results and user experience, therefore, the present disclosure adopts a feature modeling and classification method for feature extraction to determine whether the pelvic floor muscle is in motion or at rest, which can efficiently solve such problems;
performing TAG operation on the raw I/Q value training data of pelvic floor muscle signals and inputting it into the feature value calculation module to calculate the feature values. The feature values selected in the present disclosure are standard deviation, root mean square, waveform factor, and peak factor, which are calculated according to the following formula:
standard deviation:

$${X}_{s} = \sqrt{{\sum }_{i = 1}^{n} {( {x}_{i} - x )}^{2} / n} ;$$

root mean square:

$${X}_{r m s} = \sqrt{{\sum }_{i = 1}^{n} {x}^{2} / n} ;$$

waveform factor:

$${k}_{f} = {X}_{r m s} / {X}_{arv} ;$$

$$peak factor :$$

$${k}_{p} = {X}_{max} / {X}_{r m s} ;$$

wherein,

$${X}_{arv} = \frac{1}{n} {\sum }_{i = 1}^{n} {x}_{i} .$$

After calculating the feature values of the training samples, performing feature extraction and inputting the results into a classification regression tree for classification. The classification regression tree used in the present disclosure selects the Gini coefficient (GINI) as the screening criterion for feature selection. Based on the feature values, the dataset D is divided into two different categories, S and M, wherein S represents pelvic floor muscle static and M represents pelvic floor muscle dynamic. The Gini coefficient used is calculated using the following formula:

$${G}_{S , M} = {S}_{1} {G}_{{S}_{1}} ( {S}_{1} ) + \frac{{M}_{1}}{D} {G}_{{M}_{1}} ( {M}_{1} ) ;$$

when using the Gini coefficient to partition attributes, we choose the dataset with the smaller Gini index in the current attribute partition as the split subset. Therefore, the following nodes repeat this process and continuously split, and the decision tree can be generated.

If the input vector of the decision tree is X and there are J categories for classification, then the edge functions for the input vector X and output Y are defined as:

$$K ( X , Y ) = {a}_{k} I ( h ( X , {θ}_{k} ) = Y ) - max ( {a}_{k} I ( h ( X , {θ}_{k} ) = j ) ) ;$$

wherein, j is the predicted category of X by the current decision tree, I( ) is the indicator function; θₖ is the average number of bagging for other categories; k is the decision tree number. The edge function measures the confidence of correct classification, and the larger the value, the better the classification performance.

The upper bound of decision tree generalization error is shown in the following formula:

$${E}^{*} \leq \frac{ρ ( 1 - {s}^{2} )}{{s}^{2}}$$

wherein, ρ is the correlation coefficient of the decision tree, s is the average strength of the decision tree.

For this decision tree model, after sampling the training set, the Gini coefficient is selected to partition the left and right subtrees. Bagging is used for partitioning, and for each sample, the Gini coefficient is used to determine whether the pelvic floor muscle state is in motion or at rest. Then, based on the bagging results, the pelvic floor muscle movement state in that window is ultimately determined.
step five:
based on the determination results of the pelvic floor muscle state in step four, signal processing and integration are performed, and the motion waveform processed by the pelvic floor muscle is displayed in real-time by the signal transmission module.

According to the above technical solution, in step S4, adjusting the magnetic stimulation parameters based on the analysis and detection results of S3 comprising the following steps:
Z1: determining the maximum autonomous contraction intensity of the patient's pelvic floor muscle;
Z2: determining the dynamic range of effective induced magnetic stimulation parameters; using magnetic stimulation sequences with different parameter combinations to stimulate the pelvic floor muscle, and simultaneously collecting the movement state of pelvic floor muscle, when the induced pelvic floor muscle movement intensity exceeds the standard set by Z1, the current magnetic stimulation parameter is considered as the effective magnetic stimulation parameter;
Z3: adjusting the stimulation strategy and selecting the current optimal stimulation parameter from the effective magnetic stimulation parameters for magnetic stimulation during use; the stimulation strategy described in Z3 is implemented by the following methods:
Z3-1: arranging the obtained set of effective magnetic stimulation parameters, wherein the combination of stimulation parameters corresponding to the minimum stimulation intensity and minimum stimulation frequency is ranked first, and other effective magnetic stimulation parameters are sorted according to the induced pelvic floor muscle strength;
Z3-2: Real-time monitoring the pelvic floor muscle movement state by the electromagnetic waves. When the induced contraction intensity weakens, the latter stronger magnetic stimulation parameter is selected from the effective magnetic stimulation parameters until the induced contraction intensity exceeds a % of the maximum autonomous contraction again;
if the maximum stimulation intensity and frequency cannot induce the greater contraction, it indicates that the patient has entered a fatigue state, and testing should be performed on the patient after rest.

According to the above technical solution, embodiments of the method further comprising: S5: monitoring the pelvic floor movement data, and determining whether to automatically trigger the magnetic stimulation device to generate pulse magnetism based on the autonomous movement data; further strengthening the movement of the pelvic floor muscle, such that improving the effectiveness of pelvic floor muscle movement, achieving the combination of active training and passive physical therapy, and further improving the treatment effect of pelvic floor problems.

Wherein, embodiments of the method of triggering magnetic stimulation comprising the following steps:
T1: detecting the strongest autonomous contraction intensity of the patient's pelvic floor muscle, that is, detecting the amplitude of pelvic floor muscle movement generated by the patient during the strongest autonomous contraction of the pelvic floor muscle, which is used to determine the dynamic range of effective induced magnetic stimulation parameters and determine the threshold for triggering magnetic stimulation subsequently;
T2: determining the dynamic range of effective induced magnetic stimulation parameters; that is, using a magnetic stimulation sequence with different parameter combinations to stimulate the pelvic floor muscle, and simultaneously collecting the movement state of the pelvic floor muscle, only when the induced pelvic floor muscle movement intensity exceeds the standard set by T1, the current magnetic stimulation parameter is considered as the effective magnetic stimulation parameter;
T3: determining whether the patient has sufficient pelvic floor muscle contraction and trigger the magnetic stimulation strategy; if not, the patient needs to strengthen their efforts; if there is, triggering magnetic stimulation to enhance the contraction of the patient's pelvic floor muscle. When enhancing stimulation, the current optimal stimulation parameter will be prioritized from the effective magnetic stimulation parameters for magnetic stimulation. The optimal condition is for example to prioritize the magnetic stimulation with the lowest stimulation intensity and pulse frequency, under the same induction intensity, in order to delay fatigue and prolong the duration of magnetic stimulation.

The above method can be implemented in the following ways: firstly, arranging the set of effective magnetic stimulation parameters obtained from T2, wherein the combination of stimulation parameters corresponding to the minimum stimulation intensity and minimum stimulation frequency is ranked first, and the other effective magnetic stimulation parameters are sorted according to the induced pelvic floor muscle strength. At the same time, electromagnetic waves will monitor the movement state of pelvic floor muscle in real-time, when the induced contraction intensity weakens, the latter stronger magnetic stimulation parameter will be selected from the effective magnetic stimulation parameters until the induced contraction intensity exceeds 80% of the maximum autonomous contraction again. If the maximum stimulation intensity and frequency cannot induce the greater contraction, it indicates that the patient has entered a fatigue state and needs to perform testing after rest.

An apparatus for implementing the application of a non-contact pelvic floor muscle real-time detection and feedback system.

Compared with the conventional art, the beneficial effects of the present disclosure are:
1. The present disclosure does not require the sensor to be placed inside the human body, which not only protects the privacy of patients, eliminates the risk of cross infection, but also does not require consumables, reducing the cost of use.
2. The present disclosure can monitor the passive contraction of pelvic floor muscle during magnetic stimulation in real-time, thereby monitoring their effectiveness in real-time, the stimulation plan can be adjusted in real-time according to the current pelvic floor muscle state, which can improve the detection effect and patient experience.
3. The present disclosure can detect the active contraction of the patient's pelvic floor in real-time and trigger a magnetic stimulation pulse to achieve the function of triggering magnetic stimulation, it can not only strengthen the contraction of pelvic floor muscle, but also improve patient participation, the detection effect is significantly improved by combining active and passive contraction.

### BRIEF DESCRIPTION

Some of the embodiments will be described in detail, with references to the following Figures, wherein like designations denote like members, wherein:

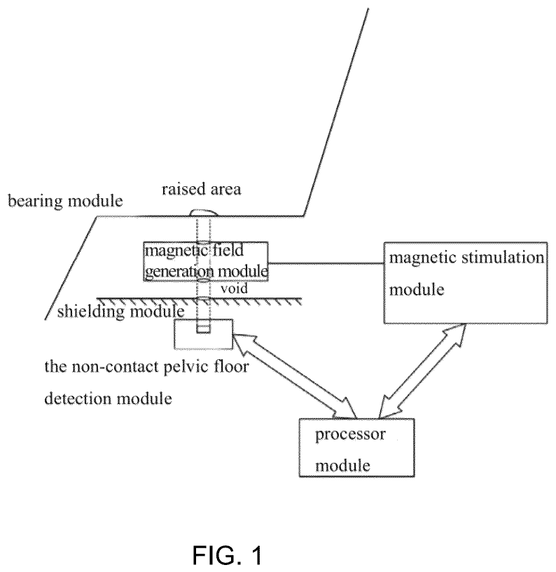

FIG. 1 shows a schematic diagram of the structure of the non-contact pelvic floor muscle real-time detection feedback system, method, and apparatus in the present disclosure;

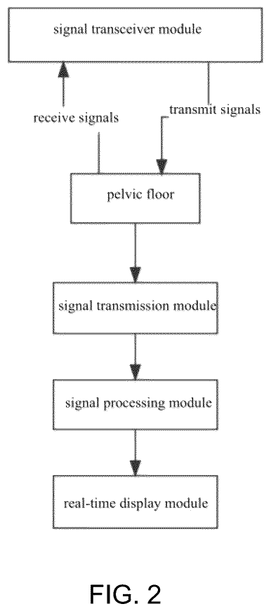

FIG. 2 shows a schematic diagram of embodiments of the system composition of the non-contact pelvic floor detection module in the present disclosure;

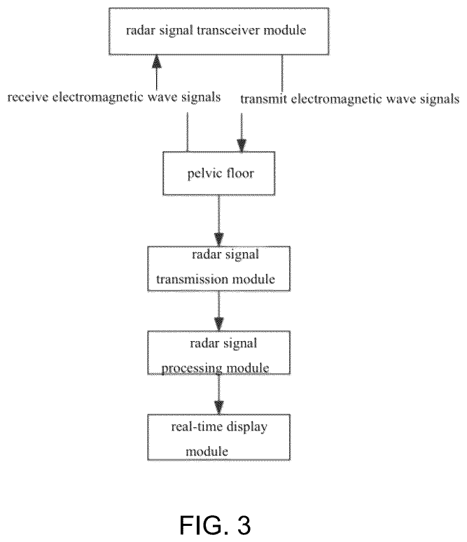

FIG. 3 shows a schematic diagram of a specific example of the non-contact pelvic floor detection module in the present disclosure;

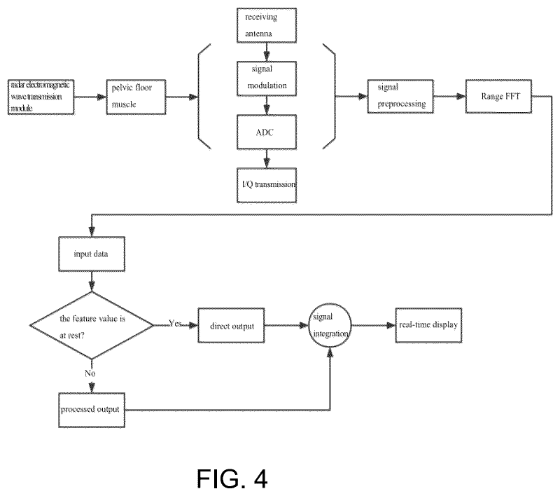

FIG. 4 shows a schematic flowchart of the signal processing method presented based on text;

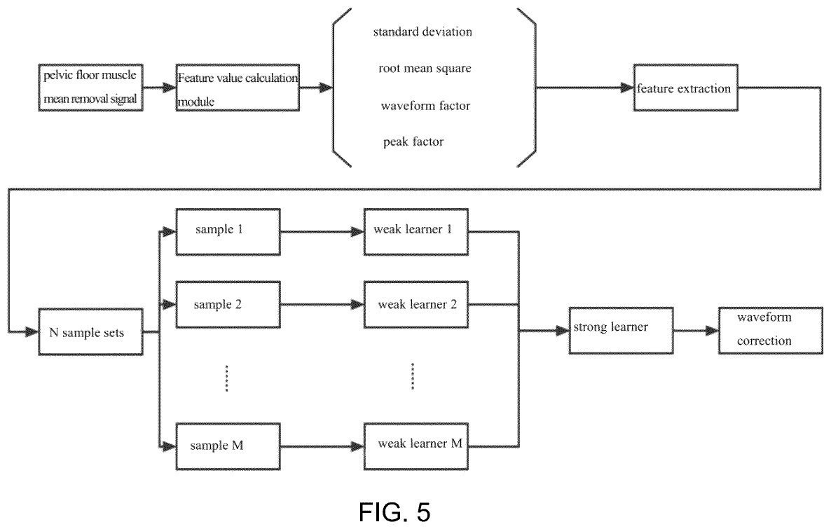

FIG. 5 shows a schematic flowchart of the specific process of the pelvic floor muscle state determination module presented based on text;

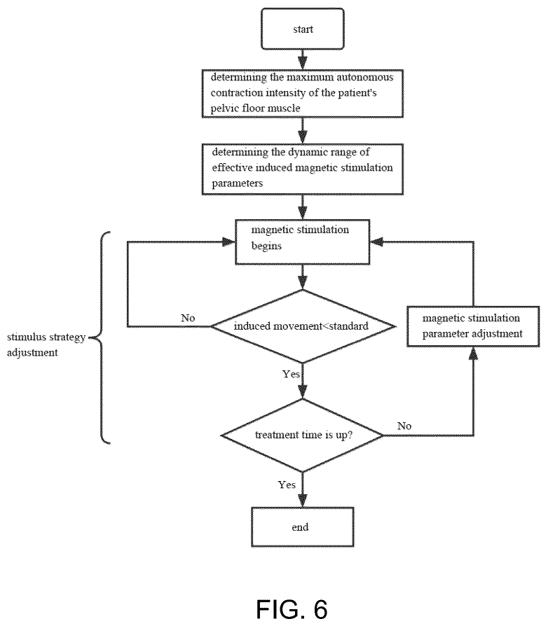

FIG. 6 shows a schematic flowchart of embodiments of the method for monitoring and real-time adjusting the magnetic stimulation parameters in the present disclosure;

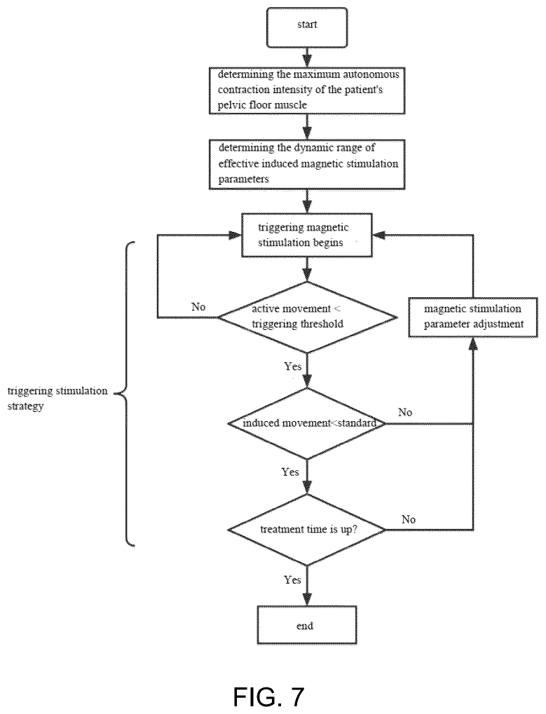

FIG. 7 shows a schematic flowchart of embodiments of the method for triggering magnetic stimulation in the present disclosure;

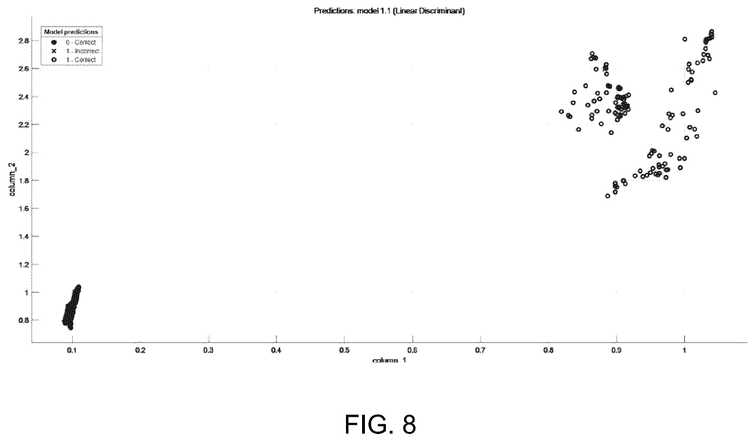

FIG. 8 shows a schematic diagram of using decision trees for motion state classification in embodiment one presented based on text; and

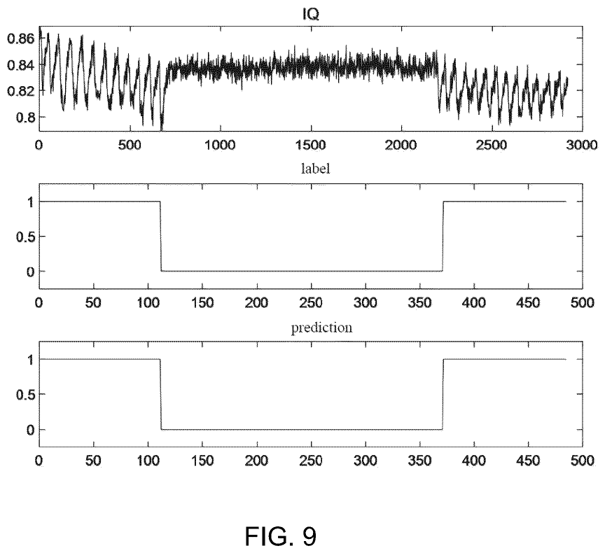

FIG. 9 shows a schematic diagram of the results of using decision trees for motion state classification in embodiment one presented based on text.

### DETAILED DESCRIPTION

The following description will clearly and completely describe the technical solutions in the embodiments of the present disclosure with reference to the accompanying drawings. It should be understood that the embodiments described herein represent only a portion of the embodiments of the present disclosure, not all of them. All other embodiments obtained by those skilled in the art based on the embodiments of the present disclosure without performing creative labor fall within the scope of protection of the present disclosure.

Referring to FIG. 1 to FIG. 9, the present disclosure provides the following technical solutions:
a non-contact pelvic floor muscle real-time detection and feedback system, method and apparatus, embodiments of the system comprising:
a processor module for controlling a magnetic stimulation module and a magnetic field generation module to generate pulse magnetic fields with different parameters;
the processor module controls the operation of the non-contact pelvic floor detection module, receives data from the non-contact pelvic floor detection module, processes the data, and acquires the pelvic floor muscle real-time state; the processor module detects the pelvic floor state in real-time when the pulse magnetic field stimulates the pelvic floor, analyzes the detected data, and adjusts the magnetic stimulation parameters based on the feedback.

Wherein, embodiments of the method for monitoring and real-time adjusting the magnetic stimulation parameters comprising the following steps:
Z1: Determining the maximum autonomous contraction intensity of the patient's pelvic floor muscle; the strongest autonomous contraction intensity detection, that is, detecting the amplitude of pelvic floor muscle movement generated by the patient during the strongest autonomous contraction of the pelvic floor muscle, which is used to determine the dynamic range of effective induced magnetic stimulation parameters subsequently, and establish personalized standards. One method comprises collecting the strongest autonomous contraction intensity as 80% of the RMS average value of the 200 ms window with within 5 seconds, as a standard for the dynamic range of subsequent effective induced magnetic stimulation parameters.
Z2: Determining the dynamic range of effective induced magnetic stimulation parameters;
wherein, using magnetic stimulation sequences with different parameter combinations to stimulate the pelvic floor muscle, and simultaneously collecting the movement state of pelvic floor muscle, when the induced pelvic floor muscle movement intensity exceeds the standard set by Z1, the current magnetic stimulation parameter is considered as the effective magnetic stimulation parameter; one specific implementation method is to adjust the magnetic stimulation intensity and magnetic stimulation pulse frequency, such as increasing the magnetic stimulation intensity from 1 T to 6 T with a step of 1 T; increasing the magnetic stimulation pulse frequency from 10 Hz to 100 Hz with a step of 10 Hz; each combination lasts for 200 ms, using this as a window; calculating the RMS value within a window, and when it exceeds 80% of the RMS standard in step one, determining it as an effective magnetic stimulation parameter. The set of all effective magnetic stimulation parameters is the dynamic range of effective induced magnetic stimulation parameters.
Z3: Adjusting the stimulation strategy and selecting the current optimal stimulation parameter from the effective magnetic stimulation parameters for magnetic stimulation during use. The optimal condition is for example to prioritize the magnetic stimulation with the lowest stimulation intensity and pulse frequency, under the same induction intensity, in order to delay fatigue and prolong the duration of magnetic stimulation.

The stimulation strategy is implemented by the following methods:
Z3-1: arranging the set of effective magnetic stimulation parameters obtained from Z2, wherein the combination of stimulation parameters corresponding to the minimum stimulation intensity and minimum stimulation frequency is ranked first, and other effective magnetic stimulation parameters are sorted according to the induced pelvic floor muscle strength;
Z3-2: When using common magnetic stimulation, starting with the optimal magnetic stimulation parameter, real-time monitoring the pelvic floor muscle movement state by the electromagnetic waves. When the induced contraction intensity weakens, the latter stronger magnetic stimulation parameter is selected from the effective magnetic stimulation parameters, until the induced contraction intensity exceeds 80% of the maximum autonomous contraction again.

If the maximum stimulation intensity and stimulation frequency cannot induce the greater contraction, it indicates that the patient has entered a fatigue state, and testing needs to be performed on the patient after rest.

The magnetic field generation module is connected to the magnetic stimulation module;
the magnetic field generation module comprises a metal coil inside, the instantaneous current generated by the magnetic stimulation module generates a strong pulse magnetic field by the magnetic field generation module, the magnetic field generation module is placed below the bearing module, the magnetic field generation module has a void in the center that facilitates the electromagnetic waves transmitted by the non-contact pelvic floor detection module to pass through.

Wherein, embodiments of the method of triggering magnetic stimulation comprising the following steps:
T1: Detecting the strongest autonomous contraction intensity of the patient's pelvic floor muscle, that is, detecting the amplitude of pelvic floor muscle movement generated by the patient during the strongest autonomous contraction of the pelvic floor muscle, which is used to determine the dynamic range of effective induced magnetic stimulation parameters and determine the threshold for triggering magnetic stimulation subsequently. One specific method is to first calculate the strongest autonomous contraction intensity as the RMS average of the 200 ms window within 5 seconds and then determine 30% of the strongest autonomous contraction intensity as the trigger threshold for magnetic stimulation.
T2: Determining the dynamic range of effective induced magnetic stimulation parameters; that is, using a magnetic stimulation sequence with different parameter combinations to stimulate the pelvic floor muscle, and simultaneously collecting the movement state of the pelvic floor muscle, only when the induced pelvic floor muscle movement intensity exceeds the standard set by T1, the current magnetic stimulation parameter is considered as the effective magnetic stimulation parameter. One method comprises adjusting the magnetic stimulation intensity and magnetic stimulation pulse frequency, such as increasing the magnetic stimulation intensity from 1 T to 6 T with a step of 1 T; increasing the magnetic stimulation pulse frequency from 10 Hz to 100 Hz with a step of 10 Hz; each combination lasts for 200 ms, using this as a window. Calculating the RMS value within a window, and when it exceeds 80% of the RMS standard in step one, determining it as an effective magnetic stimulation parameter. The set of all effective magnetic stimulation parameters is the dynamic range of effective induced magnetic stimulation parameters.
T3: Determining whether the patient has sufficient pelvic floor muscle contraction and trigger the magnetic stimulation strategy, such as whether it exceeds the threshold; if not, the patient needs to strengthen their efforts; if there is, triggering magnetic stimulation to enhance the contraction of the patient's pelvic floor muscle. When enhancing stimulation, the current optimal stimulation parameter will be prioritized from the effective magnetic stimulation parameters for magnetic stimulation. The optimal condition is for example to prioritize the magnetic stimulation with the lowest stimulation intensity and pulse frequency, under the same induction intensity, in order to delay fatigue and prolong the duration of magnetic stimulation. The above method can be implemented in the following ways: firstly, arranging the set of effective magnetic stimulation parameters obtained from T2, wherein the combination of stimulation parameters corresponding to the minimum stimulation intensity and minimum stimulation frequency is ranked first, and the other effective magnetic stimulation parameters are sorted according to the induced pelvic floor muscle strength. At the same time, electromagnetic waves will monitor the movement state of pelvic floor muscle in real-time. When the induced contraction intensity weakens, the latter stronger magnetic stimulation parameter will be selected from the effective magnetic stimulation parameters, until the induced contraction intensity exceeds 80% of the maximum autonomous contraction again. If the maximum stimulation intensity and stimulation frequency cannot induce the greater contraction, it indicates that the patient has entered a fatigue state and needs to perform testing after rest.

The non-contact pelvic floor detection module is used to detect pelvic floor muscle movements by non-contact detection methods;
the non-contact pelvic floor detection module comprises an antenna module for transmitting and receiving electromagnetic waves, which can detect the movement of pelvic floor muscles and other tissues driven by the pelvic floor, the non-contact pelvic floor detection module is placed below the magnetic field generation module, the antenna module for transmitting and receiving electromagnetic waves is aligned with the center of the void of the magnetic field generation module, the electromagnetic waves transmitted by the antenna module of the non-contact pelvic floor detection module, due to their distance from the human body, can detect all movements within their radiation area, and are easily affected by the interference from non-pelvic floor muscle movements such as thighs and buttocks, as well as the interference from environmental noise. One solution for non-contact pelvic floor detection module is millimeter wave or centimeter wave radar, such as 24 GHz radar sensor, 60 GHz radar sensor, 77 GHz radar sensor, etc. It can be located at a certain distance from the human body and can penetrate objects such as clothing, achieving non-contact pelvic floor detection.

As shown in FIG. 2, the non-contact pelvic floor detection module includes a signal transceiver module, a signal transmission module, a signal processing module, and a real-time display module;
the signal transceiver module is used to detect the pelvic floor muscle state by transmitting and receiving signals; the signal transmission module is used to transmit the received signal; including signal modulation, ADC, and I/Q transmission; the signal processing module is used to process the signal data transmitted by the signal transmission module; the real-time display module is used to real-time display the processed signal data;

As shown in FIG. 3, the signal transceiver module can use radar electromagnetic wave transceiver module for non-contact detection, including radar electromagnetic wave transmitting antenna and receiving antenna.

As shown in FIG. 4, non-contact pelvic floor detection comprising the following steps:
step one: the signal transceiver module transmits a signal, which touches the pelvic floor area, and generates an echo reflection that is received by the signal transceiver module and transmitted by the transmission module; one way is to transmit the electromagnetic waves by the radar electromagnetic wave transmission module. When the incident electromagnetic waves touch the skin tissue outside the pelvic floor muscle, they generate echoes and reflect back, which are received by the radar receiving antenna. After signal modulation, they are converted into digital signals raw data by the radar's ADC module and transmitted in the form of I/Q signals, wherein, the data format of I/Q signals is I+iQ, I is the real part and Q is the imaginary part;
step two: the signal processing module preprocesses the signals transmitted by the signal transmission module; specifically, one method is to preprocess the obtained raw signal rawdata, and after performing a Range Fourier Transform (FFT) on it, the resulting data is signal X={X₁, X₂, . . . , X_{N}}, wherein, X₁, X₂, . . . , X_{N} represent the data from 1 to N distance gates obtained by performing the Range Fourier Transform on the received signal X, representing the radar echo signal values detected from different distances, the size of N values is positively correlated with the number of distance gates;
step three: the signal processing module processes the preprocessed data and restores the true signal of pelvic floor muscle movement in the preprocessed signal; specifically, one method is sliding window filtering processing, due to the fact that the object touched by electromagnetic waves after transmission includes not only the target pelvic floor area, but also other static components such as walls and seats, in order to ensure that the radar phase changeΔ @ is positively correlated with the pelvic floor muscle movement change Δ d, it is necessary to eliminate the static component in signal X, namely: X=Aₘe^{jWm}+Aₛe^{jWs},
wherein, Aₘ represents the amplitude of the dynamic signal component, Aₛ is the amplitude of the static signal component, Wₘ is the phase of the dynamic signal component, Wₛ is the phase of the static signal component.

Selecting a sliding window for real-time signal processing, the window length WinLen needs to be set empirically based on the radar sampling rate, the Step for each slide is set as 5;
performing mean removal on the signal values within each window, i.e. X′ᵢ=Xᵢ−mean (X_{i-WinLen+1}:Xᵢ);
wherein, mean (X_{A}:X_{B}) represents the mean of the A-th to B-th data in array X.

After this method, low-frequency signal components in the signal can be filtered out.
step four:
using feature modeling and classification methods for feature extraction to determine the state of pelvic floor muscle; after the mean filtering, the low-frequency components, i.e. static component Aₛe^{jWs}, in the signal X=Aₘe^{jWm}+Aₛe^{jWs} are basically filtered out, after passing through the filter, Aₘ in the dynamic component Aₘe^{jWm} remains basically unchanged, but its phase Wₘ undergoes sudden changes, resulting in irregular changes in the waveform, therefore, the calculated phase changeΔ φ is not correlated with the pelvic floor muscle movement change Δ d.

In conventional processing methods, it is not possible to recover the signal waveform of the true movement of the pelvic floor muscle, and the waveform cannot correspond to the movement of the pelvic floor muscle, and when the pelvic floor muscle remain stationary and contracted for a long time, the filtered waveform is prone to jumping, which affects the test results and user experience. Therefore, the present disclosure adopts a feature modeling and classification method for feature extraction to determine whether the pelvic floor muscle is in motion or at rest, which can efficiently solve such problems.

Performing TAG operation on the raw I/Q value training data of pelvic floor muscle signals and inputting it into the feature value calculation module to calculate the feature values. The feature values selected in the present disclosure are standard deviation, root mean square, waveform factor, and peak factor, which are calculated according to the following formula:
standard deviation:

$${X}_{s} = \sqrt{{\sum }_{i = 1}^{n} {( {x}_{i} - x )}^{2} / n} ;$$

root mean square:

$${X}_{rms} = \sqrt{{\sum }_{i = 1}^{n} {x}^{2} / n} ;$$

waveform factor:

$${k}_{f} = {X}_{r m s} / {X}_{arv} ;$$

$$peak factor : {k}_{p} = {X}_{max} / {X}_{rms} ;$$

wherein,

$${X}_{arv} = \frac{1}{n} {\sum }_{i = 1}^{n} {x}_{i} .$$

After calculating the feature values of the training samples, performing feature extraction and inputting the results into a classification regression tree for classification. The classification regression tree used in the present disclosure selects the Gini coefficient (GINI) as the screening criterion for feature selection. Based on the feature values, the dataset D is divided into two different categories, S and M, wherein S represents pelvic floor muscle static and M represents pelvic floor muscle dynamic. The Gini coefficient used is calculated using the following formula:

$${G}_{S , M} = \frac{{S}_{1}}{D} {G}_{{S}_{1}} ( {S}_{1} ) + \frac{{M}_{1}}{D} {G}_{{M}_{1}} ( {M}_{1} ) .$$

when using the Gini coefficient to partition attributes, we choose the dataset with the smaller Gini index in the current attribute partition as the split subset. Therefore, the following nodes repeat this process and continuously split, and the decision tree can be generated.

If the input vector of the decision tree is X and there are J categories for classification, then the edge functions for the input vector X and output Y are defined as:

$$K ( X , Y ) = {a}_{k} I ( h ( X , {θ}_{k} ) = Y ) - max ( {a}_{k} I ( h ( X , {θ}_{k} ) = j ) ) ;$$

wherein, j is the predicted category of X by the current decision tree, I( ) is the indicator function; θₖ is the average number of bagging for other categories; k is the decision tree number. The edge function measures the confidence of correct classification, and the larger the value, the better the classification performance.

The upper bound of decision tree generalization error is shown in the following formula:

$${E}^{*} \leq \frac{ρ ( 1 - {s}^{2} )}{{s}^{2}}$$

wherein, ρ is the correlation coefficient of the decision tree, s is the average strength of the decision tree.

For this decision tree model, after sampling the training set, the Gini coefficient is selected to partition the left and right subtrees. Bagging is used for partitioning, and for each sample, the Gini coefficient is used to determine whether the pelvic floor muscle state is in motion or at rest. Then, based on the bagging results, the pelvic floor muscle movement state in that window is ultimately determined.
step five:
based on the determination results of the pelvic floor muscle state in step four, signal processing and integration are performed, and the motion waveform processed by the pelvic floor muscle is displayed in real-time by the signal transmission module.

The bearing module is used to carry patients; there is a raised area above the bearing module. The raised area is an area where magnetic stimulation is concentrated and is also a detection area of the pelvic floor detection module. At the same time, the raised area of the bearing module can play the role of indicating positioning and adjust the posture after the patient sits up, such that making the perineum feel the raised area, realizing the function of magnetic stimulation to accurately stimulate the pelvic floor area and the function of the detection module to accurately detect the pelvic floor area. One solution is that the bearing module can be a sofa seat, and the raised area can be a silicone protrusion or an airbag protrusion in order to improve comfort.

A shielding module is used to shield the electromagnetic waves; the shielding module is placed between the non-contact pelvic floor detection module and the bearing module and is used to shield the electromagnetic waves transmitted by the non-contact pelvic floor detection module;
the shielding module has a void in the center that allows some electromagnetic waves to pass through;
wherein, the antenna module of the non-contact pelvic floor detection module, the void of the shielding module, the void of the magnetic field generation module, and the raised area of the bearing module are located on a straight line. The electromagnetic waves transmitted and received by the antenna module propagate in the space set by this straight line, and the electromagnetic waves propagated to other areas are shielded by the shielding module. Realizing the detection of specific areas of the human pelvic floor, reducing the interference from non-pelvic floor movements and the interference from complex electromagnetic environments. One solution for the shielding module is to use wave-absorbing foam material, which only allows electromagnetic waves to pass through the void area of the shielding module, and absorbs and shields electromagnetic waves that propagate to other areas.

Embodiment one: A non-contact pelvic floor muscle real-time detection feedback system is used to perform non-contact pelvic floor detection on patients. Based on the raised position of the bearing module, the patient adjusts their sitting posture autonomously and accurately stimulates the pelvic floor area by magnetic stimulation. The processor module controls the magnetic stimulation module and magnetic field generation module to generate pulse magnetic fields with different parameters. By determining the maximum autonomous contraction intensity of the patient's pelvic floor muscles and the dynamic range of effective induced magnetic stimulation parameters, monitoring and real-time adjusting magnetic stimulation parameters are achieved; controlling the non-contact pelvic floor detection module by the processor module to obtain the pelvic floor muscle state.

The signal is transmitted via the signal transceiver module. When the signal touches the pelvic floor area, it generates an echo reflection that is received by the signal transceiver module and is transmitted by the transmission module. The signal transmitted by the signal transmission module is preprocessed by the signal processing module. The preprocessed data is performed signal processing by the signal processing module, and the real signal of pelvic floor muscle movement is restored in the preprocessed signal. Feature extraction is conducted using embodiments of the method of feature modeling and classification, for each sample, the Gini coefficient is used to determine whether the pelvic floor muscle state is in motion state or at rest state. Then, based on the bagging results, the pelvic floor muscle state in this window is ultimately determined. The partition results are shown in FIG. 8, wherein the light color represents the motion state of pelvic floor muscle, and the dark color represents the rest state of pelvic floor muscle. The pelvic floor muscle state determination module of the present disclosure can distinguish pelvic floor muscle states by extracting feature values and performing classification.

After inputting the training set into the decision tree for unsupervised parameter adjustment, the modulus values of the original I/Q values of the test set are input into the decision tree. The modulus values of the original I/Q values, labeling results and prediction results are shown in FIG. 9. The results indicate that the pelvic floor muscle state determination module of the present disclosure can accurately determine whether the pelvic floor muscle is in motion state or at rest state and correct the waveform according to the actual state. Wherein, the motion state of the pelvic floor muscle is labeled as 1, and the rest state is labeled as 0.

Although the invention has been illustrated and described in greater detail with reference to the exemplary embodiment, the invention is not limited to the examples disclosed, and further variations can be inferred by a person skilled in the art, without departing from the scope of protection of the invention.

For the sake of clarity, it is to be understood that the use of “a” or “an” throughout this application does not exclude a plurality, and “comprising” does not exclude other steps or elements.

1. A non-contact pelvic floor muscle real-time detection and feedback system, wherein comprising:
a processor module for controlling a magnetic stimulation module and a magnetic field generation module to generate pulse magnetic fields with different parameters;
a magnetic field generation module for generating pulse magnetic fields;
the magnetic field generation module has a void in the center for allowing the electromagnetic waves transmitted by a non-contact pelvic floor detection module to pass through;
a non-contact pelvic floor detection module for detecting pelvic floor muscle movements by non-contact detection methods of transmitting and receiving the electromagnetic waves;
the non-contact pelvic floor detection module is placed below the magnetic field generation module, the transmitted and received electromagnetic waves pass through the void of the magnetic field generation module.

2. The non-contact pelvic floor muscle real-time detection and feedback system of claim 1, wherein the non-contact pelvic floor detection module is equipped with an antenna module for transmitting electromagnetic wave signals; the non-contact pelvic floor detection module comprising a signal transceiver module, a signal transmission module, and a signal processing module;
the signal transceiver module is used to detect the pelvic floor muscle state by transmitting and receiving signals;
the signal transmission module is used to transmit the received signal;
the signal processing module is used to process the signal data transmitted by the signal transmission module.

3. The non-contact pelvic floor muscle real-time detection and feedback system of claim 1, wherein the magnetic field generation module comprises a metal coil inside, the instantaneous current generated by the magnetic stimulation module generates a pulse magnetic field through the magnetic field generation module.

4. The non-contact pelvic floor muscle real-time detection and feedback system of claim 1, wherein the processor module controls the operation of the non-contact pelvic floor detection module, receives data from the non-contact pelvic floor detection module, processes the data, and acquires the pelvic floor muscle real-time state; the processor module detects the pelvic floor state in real-time when the pulse magnetic field stimulates the pelvic floor, analyzes the detected data, and adjusts the magnetic stimulation parameters based on the feedback.

5. The non-contact pelvic floor muscle real-time detection and feedback system of claim 1, wherein further comprising:
a bearing module for carrying patients, the bearing module is located above the magnetic field generation module; the bearing module has a raised area; the raised area is an area where magnetic stimulation is concentrated and is also a detection area of the pelvic floor detection module; the raised area can indicate to the patients and enable the patients to autonomously adjust and align with the corresponding area of the pelvic floor;
a shielding module for shielding the electromagnetic waves; the shielding module is placed between the non-contact pelvic floor detection module and the magnetic field generation module, and the shielding module has a void in the center;
the non-contact pelvic floor detection module, the void of the shielding module, the void of the magnetic field generation module, and the raised area of the bearing module are located on a straight line, the electromagnetic waves transmitted and received by the antenna module propagate in the void, and the electromagnetic waves propagated to other areas are shielded by the shielding module.

6. A non-contact pelvic floor muscle real-time detection and feedback method, comprising:
S1: generating instantaneous current by a magnetic stimulation module and generating a pulse magnetic field by a magnetic field generation module;
S2: performing a non-contact detection of the pelvic floor by transmitting and receiving electromagnetic waves through a non-contact pelvic floor detection module;
S3: acquiring the detected pelvic floor state data by a processor module, and performing analysis and detection on it;
S4: adjusting-magnetic stimulation parameters based on the analysis and detection results from S3.

7. The non-contact pelvic floor muscle real-time detection and feedback method of claim 6, wherein in steps S2 and S3, the non-contact pelvic floor detection module comprises a signal transceiver module, a signal transmission module, and a signal processing module, performing the non-contact pelvic floor detection and analyzing the pelvic floor state comprising the following steps:
step one:
using the signal transceiver module to transmit a signal, the signal touches the pelvic floor area and generates an echo reflection that is received by the signal transceiver module again and is transmitted to the signal processing module by the transmission module;
step two:
using the signal processing module to perform a Range Fourier Transform on the received reflected signal, the resulting data is signal X={X₁, X₂, . . . , X_{N}}, wherein, X₁, X₂, . . . , X_{N} represent the data from 1 to N distance gates obtained by performing the Range Fourier Transform on the received signal X, representing the radar echo signal values detected from different distances, the size of N values is positively correlated with the number of distance gates;
step three:
using the signal processing module to restore the true signal of pelvic floor muscle movement;
remove the static component from signal X: X=Aₘe^{jWm}+Aₛe^{jWs}, wherein, Aₘ represents the amplitude of the dynamic signal component, Aₛ is the amplitude of the static signal component, Wₘ is the phase of the dynamic signal component, Wₛ is the phase of the static signal component;
selecting a sliding window for real-time signal processing, the window length WinLen needs to be set empirically based on the radar sampling rate;
performing mean removal on the signal values within each window:
$${X}_{i}^{′} = {X}_{i} - mean ( {X}_{i - WinLen + 1} : {X}_{i} ) ;$$
wherein, mean (X_{A}:X_{B}) represents the mean of the A-th to B-th data in array X;
step four:
using feature modeling and classification methods for feature extraction to determine the state of pelvic floor muscle; performing TAG operation on the raw I/Q value training data of pelvic floor muscle signals and inputting it into the feature value calculation module to calculate the feature values, performing feature extraction on the feature value of the calculated training samples, and inputting the results into a classification regression tree for classification to determine the pelvic floor muscle state;
step five:
based on the determination results of the pelvic floor muscle state in step four, signal processing and integration are performed, and the motion waveform processed by the pelvic floor muscle is displayed in real-time by the signal transmission module.

8. The non-contact pelvic floor muscle real-time detection and feedback method of claim 7, wherein in step four, the selected feature values are standard deviation, root mean square, waveform factor, and peak factor, which are calculated according to the following formula:
standard deviation:
$${X}_{s} = \sqrt{{\sum }_{i = 1}^{n} {( {x}_{i} - x )}^{2} / n} ;$$
root mean square:
$${X}_{rms} = \sqrt{{\sum }_{i = 1}^{n} {x}^{2} / n} ;$$
waveform factor:
$${k}_{f} = {X}_{r m s} / {X}_{arv} ;$$
$$peak factor : {k}_{p} = {X}_{max} / {X}_{rms} ;$$
wherein,
$${X}_{arv} = \frac{1}{n} {\sum }_{i = 1}^{n} {x}_{i} ;$$
calculating the feature values of the training samples, performing feature extraction and inputting the results into a classification regression tree for classification;
wherein, the classification regression tree selects the Gini coefficient as the screening criterion for feature selection, based on the feature values, the dataset D is divided into two different categories of datasets, S and M, wherein S represents pelvic floor muscle static and M represents pelvic floor muscle dynamic, the Gini coefficient used is calculated using the following formula:
$${G}_{S , M} = \frac{{S}_{1}}{D} {G}_{{S}_{1}} ( {S}_{1} ) + \frac{{M}_{1}}{D} {G}_{{M}_{1}} ( {M}_{1} ) ;$$
when using the Gini coefficient to partition attributes, choosing the dataset with the smaller Gini index in the current attribute partition as the split subset, the following nodes repeat this process and continuously split to generate the decision tree;
if the input vector of the decision tree is X and there are J categories for classification, then the edge functions for the input vector X and output Y are defined as:
$$K ( X , Y ) = {a}_{k} I ( h ( X , {θ}_{k} ) = Y ) - max ( {a}_{k} I ( h ( X , {θ}_{k} ) = j ) ) ;$$
wherein, j is the predicted category of X by the current decision tree, I( ) is the indicator function;
θₖ is the average number of bagging for other categories; k is the decision tree number;
the upper bound of decision tree generalization error is shown in the following formula:
$${E}^{*} \leq \frac{ρ ( 1 - {s}^{2} )}{{s}^{2}} ;$$
wherein, ρ is the correlation coefficient of the decision tree, s is the average strength of the decision tree;
for this decision tree model, after sampling the training set, the Gini coefficient is selected to partition the left and right subtrees, bagging is used for partitioning, and for each sample, the Gini coefficient is used to determine whether the pelvic floor muscle state is in motion or at rest, and then based on the bagging results, the pelvic floor muscle movement state in this window is determined.

9. The non-contact pelvic floor muscle real-time detection and feedback method of claim 6, wherein in step S4, adjusting the magnetic stimulation parameters based on the analysis and detection results of S3 comprising the following steps:
Z1: determining the maximum autonomous contraction intensity of the patient's pelvic floor muscle;
Z2: determining the dynamic range of effective induced magnetic stimulation parameters; using magnetic stimulation sequences with different parameter combinations to stimulate the pelvic floor muscle, and simultaneously collecting the movement state of pelvic floor muscle, when the induced pelvic floor muscle movement intensity exceeds the standard set by Z1, the current magnetic stimulation parameter is considered as the effective magnetic stimulation parameter;
Z3: adjusting the stimulation strategy and selecting the current optimal stimulation parameter from the effective magnetic stimulation parameters for magnetic stimulation during use.

10. The non-contact pelvic floor muscle real-time detection and feedback method of claim 9, wherein the stimulation strategy in Z3 is implemented by the following methods:
Z3-1: arranging the obtained set of effective magnetic stimulation parameters, wherein the combination of stimulation parameters corresponding to the minimum stimulation intensity and minimum stimulation frequency is ranked first, and other effective magnetic stimulation parameters are sorted according to the induced pelvic floor muscle strength;
Z3-2: Real-time monitoring the pelvic floor muscle movement state by the electromagnetic waves, when the induced contraction intensity weakens, the latter stronger magnetic stimulation parameter is selected from the effective magnetic stimulation parameters, until the induced contraction intensity exceeds a % of the maximum autonomous contraction again;
if the maximum stimulation intensity and stimulation frequency cannot induce the greater contraction, it indicates that the patient has entered a fatigue state, and testing should be performed on the patient after rest.

11. The non-contact pelvic floor muscle real-time detection and feedback method of claim 6, wherein the method further comprising: S5: monitoring the pelvic floor movement data, and determining whether to automatically trigger the magnetic stimulation device to generate pulse magnetism based on the autonomous movement data;
wherein, the method of triggering magnetic stimulation comprising the following steps:
T1: detecting the strongest autonomous contraction intensity of the patient's pelvic floor muscle, which is used to determine the dynamic range of effective induced magnetic stimulation parameters and determine the threshold for triggering magnetic stimulation subsequently;
T2: determining the dynamic range of effective induced magnetic stimulation parameters; when the induced pelvic floor muscle movement intensity exceeds the standard set by T1, the current magnetic stimulation parameter is considered as the effective magnetic stimulation parameter;
T3: determining whether to trigger the magnetic stimulation strategy based on the degree of patients' pelvic floor muscle contraction.

12. An apparatus, wherein the application of a non-contact pelvic floor muscle real-time detection feedback system of claim 1 on the apparatus.
